(19) United States
(12) Patent Application Publication
(10) Pub. No.: US 2023/0190292 A1
(43) Pub. Date: Jun. 22, 2023

(54) OCCLUSIVE DEVICES WITH PETAL-SHAPED REGIONS FOR TREATING VASCULAR DEFECTS

(71) Applicant: Covidien LP, Mansfield, MA (US)

(72) Inventors: Animesh Choubey, Chino Hills, CA (US); Robert A. Pecor, Aliso Viejo, CA (US); Behraam Hussain Baqai, Irvine, CA (US); Jason Chun-Yip Kwok, Anaheim, CA (US); Mehdi Rashidi, Irvine, CA (US)

(21) Appl. No.: 18/066,811

(22) Filed: Dec. 15, 2022

Related U.S. Application Data
(60) Provisional application No. 63/265,564, filed on Dec. 16, 2021.

(51) Int. Cl. A61B 17/12

(52) U.S. Cl. CPC A61B 17/12113; A61B 17/12172; A61B 2017/12054

(57) ABSTRACT

Devices for treating vascular defects and associated systems and methods are disclosed herein. In some embodiments, for example, an occlusive device for treating an aneurysm includes a first mesh formed from a first tubular braid, the first mesh including a first petal-shaped region formed from a first flattened section of the first tubular braid. The first mesh can be coupled to a second mesh formed from a second tubular braid. The second mesh can include a second petal-shaped region formed from a second flattened section of the second tubular braid. When the occlusive device is deployed within the aneurysm, the first and second petal-shaped regions can extend at least partially over a neck of the aneurysm.

### CROSS REFERENCE TO RELATED APPLICATION(S)

The present application claims the benefit of priority to U.S. Provisional Pat. Application No. 63/265,564, filed Dec. 16, 2021, which is incorporated by reference herein in its entirety.

### TECHNICAL FIELD

The present technology generally relates to medical devices, and in particular, to occlusive devices with petal-shaped regions for treating vascular defects.

### BACKGROUND

Intracranial saccular aneurysms occur in 1% to 2% of the general population and account for approximately 80% to 85% of non-traumatic subarachnoid hemorrhages. Recent studies show a case fatality rate of 8.3% to 66.7% in patients with subarachnoid hemorrhage. Endovascular treatment of intracranial aneurysms with coil embolization involves packing the aneurysm sac with metal coils to reduce or disrupt the flow of blood into the aneurysm, thereby enabling a local thrombus or clot to form which fills and ultimately closes off the aneurysm. Although coiling has proven to have better outcomes than surgical clipping for both ruptured and unruptured aneurysms, treating complex aneurysms using conventional coiling is challenging. This is especially true for wide-necked aneurysms because coil segments may protrude from the aneurysm sac through the neck of the aneurysm and into the parent vessel, causing serious complications for the patient.

To address this, some treatments include temporarily positioning a balloon within the parent vessel across the neck of the aneurysm to prevent the coils from migrating across the neck during delivery. Alternatively, some treatments include permanently positioning a neck-bridging stent within the parent vessel across the neck of the aneurysm to prevent the coils from migrating across the neck during delivery. While balloon-assisted or stent-assisted coiling for wide-neck aneurysms has shown better occlusion rates and lower recurrence than coiling alone, the recanalization rate of treated large/giant aneurysms can be as high as 18.2%. Moreover, the addition of a balloon or stent and its associated delivery system to the procedure increases the time, cost, and complexity of treatment. Deployment of the stent or balloon during the procedure also greatly increases the risk of an intraprocedural clot forming, and can damage the endothelial lining of the vessel wall. Permanently positioning a stent within the parent vessel increases the chronic risk of clot formation on the stent itself and associated ischemic complications, and thus necessitates the use of dual antiplatelet therapy (“DAPT”). DAPT, in turn, increases the risk and severity of hemorrhagic complications in patients with acutely ruptured aneurysms or other hemorrhagic risks. Thus, neck-bridging stents are not indicated for the treatment of ruptured aneurysms.

The above-noted drawbacks associated with balloon- and stent-assisted coiling techniques influenced the development of intraluminal flow diverting stents, or stent-like structures implanted in the parent vessel across the neck of the aneurysm that redirect blood flow away from the aneurysm, thereby promoting aneurysm thrombosis. Flow diverters have been successfully used for treating wide-neck, giant, fusiform, and blister-like aneurysms. However, because they are positioned in the parent vessel, flow diverters require DAPT to avoid clot formation on the stent itself and ischemic complications. This, in turn, increases the risk and severity of hemorrhagic complications in patients with acutely ruptured aneurysms or other hemorrhagic risks. Thus, flow diverters are not indicated for the treatment of ruptured aneurysms. Flow diverters have also shown limited efficacy in treating bifurcation aneurysms (35-50%).

Endosaccular flow disrupting devices have the potential to provide the intra-aneurysmal flow disruption of coiling with the definitive remodeling at the aneurysm-parent vessel interface achieved by intraluminal flow diverters. Endosaccular devices can be mesh devices configured to be deployed completely within the aneurysm sac, with the interstices of the mesh covering the aneurysm neck and reconstructing the aneurysm-parent vessel interface. The implant disrupts the blood flow entering and exiting the aneurysm sac (resulting in stasis and thrombosis) and supports neoendothelial overgrowth without requiring DAPT (unlike endoluminal flow diverters). Thus, endosaccular devices can be used to treat wide-necked aneurysms and ruptured aneurysms. Moreover, because the device is placed completely within the aneurysm sac, the parent and branch vessels are unimpeded and can be accessed for any further retreatment or subsequent deployment of adjunctive devices during treatment.

Accordingly, there is a need for improved devices, systems, and methods for treating aneurysms and other vascular defects.

### SUMMARY

The subject technology is illustrated, for example, according to various aspects described below. These are provided as examples and do not limit the subject technology.

In one aspect of the present technology, an occlusive device for treating an aneurysm is provided. The occlusive device can include a first mesh formed from a first tubular braid, the first mesh including a first petal-shaped region formed from a first flattened section of the first tubular braid. The occlusive device can include a second mesh coupled to the first mesh. The second mesh can be formed from a second tubular braid and can include a second petal-shaped region formed from a second flattened section of the second tubular braid. When the occlusive device is deployed within the aneurysm, the first and second petal-shaped regions can be configured to extend at least partially over a neck of the aneurysm.

In some embodiments, the first mesh includes a first tip connected to the first petal-shaped region, and the second mesh includes a second tip connected to the second petal-shaped region. The first and second tips can be rounded. The first and second tips can be made of a polymeric material.

In some embodiments, the first mesh includes a first arm region connected to the first petal-shaped region, and the second mesh includes a second arm region connected to the second petal-shaped region. When the occlusive device is deployed within the aneurysm, the first and second arm regions can be configured to engage a wall of the aneurysm. The first arm region can be formed from a first radially compressed section of the first tubular braid, and the second arm region can be formed from a second radially compressed section of the second tubular braid.

In some embodiments, the first and second tubular braids are the same tubular braid. The occlusive device can further include a bridge region connecting the first petal-shaped region and the second petal-shaped region. The bridge region can be configured to engage a wall of the aneurysm. The bridge region can be formed from a radially compressed section of the tubular braid or can be formed from a radially expanded section of the tubular braid.

In some embodiments, the first and second meshes each have a low-profile state for intravascular delivery to the aneurysm, and an expanded state for deployment within the aneurysm.

In some embodiments, the first petal-shaped region has a preset curve oriented in a first direction, and the second petal-shaped region has a preset curve oriented in a second direction. The preset curves of the first and second petal-shaped regions can be configured to bias at least a portion of the first and second petal-shaped regions against a wall of the aneurysm. The first petal-shaped region can include a proximal portion configured to cover the neck of the aneurysm, and a distal portion configured to engage a wall of the aneurysm. The second petal-shaped region can include a proximal portion configured to cover the neck of the aneurysm, and a distal portion configured to engage the wall of the aneurysm.

In some embodiments, the occlusive device further includes a first collar disposed around the first mesh distal to the first petal-shaped region, and a second collar disposed around the second mesh distal to the second petal-shaped region.

In some embodiments, the first mesh includes a first proximal end region connected to the first petal-shaped region, the second mesh includes a second proximal end region connected to the second petal-shaped region. The first and second meshes can be coupled to each other at the first and second proximal end regions. The occlusive device can further include a band coupling the first and second proximal end regions to each other. The band can include a radiopaque material.

In some embodiments, the occlusive device further includes a detachment element configured to releasably couple the first and second meshes to a pusher member.

In some embodiments, the occlusive device further includes at least one third mesh coupled to the first and second meshes. The at least one third mesh can be formed from at least one third tubular braid. The at least one third mesh can include a third petal-shaped region formed from a third flattened section of the at least one third tubular braid.

In another aspect of the present technology, a method for treating an aneurysm is provided. The method can include deploying an occlusive device into the aneurysm, the occlusive device including a first mesh formed from a first tubular braid and a second mesh formed from a second tubular braid. The deploying can include positioning a first petal-shaped region of the first mesh and a second petal-shaped region of the second mesh at least partially over a neck of the aneurysm. The method can also include introducing an embolic element into the aneurysm. The first and second petal-shaped regions can at least partially obstruct the embolic element from protruding into a parent vessel of the aneurysm.

In some embodiments, the first-petal shaped region is formed from a first flattened section of the first tubular braid, and the second petal-shaped region is formed from a second flattened section of the second tubular braid. The first mesh can include a first tip connected to the first petal-shaped region, and the second mesh can include a second tip connected to the second petal-shaped region.

In some embodiments, the first petal-shaped region has a preset curve oriented in a first direction, and the second petal-shaped region has a preset curve oriented in a second direction. The method can further include engaging a wall of the aneurysm with a distal portion of the first and second petal-shaped regions.

In some embodiments, the method further includes introducing the occlusive device to the aneurysm via a lumen of a first elongated member. The first and second meshes can be in a low-profile state when the occlusive device is in the first elongated member, and an expanded state when the occlusive device is deployed in the aneurysm. Introducing the occlusive device can include pushing the occlusive device through the lumen of the first elongated member and into the aneurysm via a pusher member. The method can also include detaching the occlusive device from the pusher member.

In some embodiments, the embolic element is introduced into the aneurysm via a lumen of a second elongated member. The method can further include positioning a distal tip portion of the second elongated member into a portion of the neck of the aneurysm that is unobstructed by the first and second petal-shaped regions. The embolic element can include a coil.

In another aspect of the present technology, an occlusive device for treating an aneurysm is provided. The occlusive device can include a first mesh including a first petal-shaped region and a first arm region. The first arm region can have a curved shape that extends distally from the first petal-shaped region. The occlusive device can also include a second mesh coupled to the first mesh, the second mesh including a second petal-shaped region and a second arm region. The second arm region can have a curved shape that extends distally from the second petal-shaped region. When the occlusive device is deployed within the aneurysm, the first and second petal-shaped regions can be configured to extend at least partially over a neck of the aneurysm, and the first and second arm regions can be configured to engage a wall of the aneurysm.

In some embodiments, the first and second meshes each have a low-profile state for intravascular delivery to the aneurysm, and an expanded state for deployment within the aneurysm. The first mesh can include a first tubular braid, and the second mesh can include a second tubular braid. The first tubular braid can include a flattened region forming the first petal-shaped region, and a radially compressed region forming the first arm region. The second tubular braid can include a flattened region forming the second petal-shaped region, and a radially compressed region forming the second arm region. Alternatively, the first and second meshes can be formed from a single tubular braid.

In some embodiments, the first petal-shaped region has a preset curve oriented in a first direction, and the first arm region has a preset curve oriented in a second direction different from the first direction. The second petal-shaped region can have a preset curve oriented in a third direction, and the second arm region can have a preset curve oriented in a fourth direction different from the third direction. When the occlusive device is deployed within the aneurysm, the first and second petal-shaped regions can each define a concave surface facing away from the neck of the aneurysm. When the occlusive device is deployed within the aneurysm, the curved shape of the first arm region can bias a distal portion of the first arm region against the wall of the aneurysm, and the curved shape of the second arm region can bias a distal portion of the second arm region against the wall of the aneurysm.

In some embodiments, the occlusive device further includes a first collar disposed around the first mesh between the first petal-shaped region and the first arm region, and a second collar disposed around the second mesh between the second petal-shaped region and the second arm region.

In some embodiments, the first mesh includes a first proximal end region connected to the first petal-shaped region, the second mesh includes a second proximal end region connected to the second petal-shaped region, and the first and second meshes are coupled to each other at the first and second proximal end regions. The occlusive device can further include a band coupling the first and second proximal end regions to each other. The band can include a radiopaque material.

In some embodiments, the occlusive device further includes a detachment element configured to releasably couple the first and second meshes to a pusher member.

In some embodiments, the occlusive device further includes at least one third mesh coupled to the first and second meshes. The at least one third mesh can include a third petal-shaped region and a third arm region, the third arm region having a curved shape that extends distally from the third petal-shaped region.

In another aspect of the present technology, an occlusive device for treating an aneurysm is provided. The occlusive device can include a first tubular braid including a first flattened region connected to a first radially compressed region, the first radially compressed region having a preset curve. The occlusive device can also include a second tubular braid coupled to the first tubular braid, the second tubular braid including a second flattened region connected to a second radially compressed region, the second radially compressed region having a preset curve. When the occlusive device is deployed within the aneurysm, the first and second flattened regions can be configured to at least partially cover a neck of the aneurysm, and the first and second radially compressed regions can be biased into engagement with the wall of aneurysm by their respective preset curves.

In some embodiments, the first and second tubular braids each have a low-profile state for intravascular delivery to the aneurysm, and an expanded state for deployment within the aneurysm. When the occlusive device is deployed within the aneurysm, the first and second flattened regions can each have a concave surface facing away from the neck of the aneurysm. The first flattened region can have a preset curve oriented in a different direction than the preset curve of the first radially compressed region, and the second flattened region can have a preset curve oriented in a different direction than the preset curve of the second radially compressed region.

In some embodiments, the occlusive device further includes a first collar disposed around the first tubular braid between the first flattened region and the first radially compressed region, and a second collar disposed around the second tubular braid between the second flattened region and the second radially compressed region.

In some embodiments, the first tubular braid includes a first proximal end region connected to the first flattened region, the second tubular braid includes a second proximal end region connected to the second flattened region, and the first and second tubular braids are coupled to each other at the first and second proximal end regions.

In some embodiments, the occlusive device further includes at least one third tubular braid coupled to the first and second tubular braids. The at least one third tubular braid can include a third flattened region connected to a third radially compressed region, the third radially compressed region having a preset curve.

In a further aspect of the present technology, a method for treating an aneurysm is provided. The method can include deploying an occlusive device into the aneurysm, the occlusive device including a first mesh and a second mesh. The deploying can include positioning a first petal-shaped region of the first mesh and a second petal-shaped region of the second mesh at least partially over a neck of the aneurysm, and engaging a wall of the aneurysm with a first arm region of the first mesh and a second arm region of the second mesh. The method can further include introducing an embolic element into the aneurysm, such that the first and second petal-shaped regions at least partially obstruct the embolic element from protruding into a parent vessel of the aneurysm.

In some embodiments, the first arm region has a curved shape that extends distally from the first petal-shaped region, and the second arm region has a curved shape that extends distally from the second petal-shaped region. The first petal-shaped region can have a preset curve oriented in a first direction, and the first arm region can have a preset curve oriented in a second direction different from the first direction. The second petal-shaped region can have a preset curve oriented in a third direction, and the second arm region can have a preset curve oriented in a fourth direction different from the third direction.

In some embodiments, the method further includes introducing the occlusive device to the aneurysm via a lumen of a first elongated member. The first and second meshes can be in in a low-profile state when the occlusive device is in the first elongated member, and an expanded state when the occlusive device is deployed in the aneurysm. Introducing the occlusive device can include pushing the occlusive device through the lumen of the first elongated member and into the aneurysm via a pusher member. The method can further include detaching the occlusive device from the pusher member.

In some embodiments, the first mesh includes a first tubular braid, and the second mesh includes a second tubular braid. The first tubular braid can include a flattened region forming the first petal-shaped region, and a radially compressed region forming the first arm region. The second tubular braid can include a flattened region forming the second petal-shaped region, and a radially compressed region forming the second arm region. In some embodiments, the first and second meshes are formed from a single tubular braid.

In some embodiments, the first arm region has a curved shape that biases a distal portion of the first arm region against the wall of the aneurysm, and the second arm region has a curved shape that biases a distal portion of the second arm region against the wall of the aneurysm.

In some embodiments, the embolic element is introduced into the aneurysm via a lumen of a second elongated member. The method can include positioning a distal tip portion of the second elongated member into a portion of the neck of the aneurysm that is unobstructed by the first and second petal-shaped regions. In some embodiments, the embolic element includes a coil.

Additional features and advantages of the present technology will be set forth in the description below, and in part will be apparent from the description, or may be learned by practice of the present technology. The advantages of the present technology will be realized and attained by the structure particularly pointed out in the written description and claims hereof as well as the appended drawings.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a perspective view of an occlusive device in an expanded state configured in accordance with embodiments of the present technology.

FIG. 1B is a top view of an individual mesh structure of the occlusive device of FIG. 1A in a flattened state.

FIG. 2A is a perspective view of another occlusive device in an expanded state configured in accordance with embodiments of the present technology.

FIG. 2B is a top view of an individual mesh structure of the occlusive device of FIG. 2A in a flattened state.

FIG. 2C is a top view of the occlusive device of FIG. 2A in an expanded state.

FIG. 3A is a perspective view of yet another occlusive device in an expanded state configured in accordance with embodiments of the present technology.

FIG. 3B is a top view of a mesh structure of the occlusive device of FIG. 3A in a flattened state.

FIG. 3C is a perspective view of another occlusive device in an expanded state configured in accordance with embodiments of the present technology.

FIG. 3D is a top view of a mesh structure of the occlusive device of FIG. 3C in a flattened state.

FIG. 4A is a side cross-sectional view of an occlusive device being deployed in an aneurysm, in accordance with embodiments of the present technology.

FIG. 4B is a side cross-sectional view of the occlusive device of FIG. 4A when fully deployed in the aneurysm, in accordance with embodiments of the present technology.

FIG. 4C is a top cross-sectional view of FIG. 4B.

FIG. 4D is a side cross-sectional view of a coil being deployed in combination with the occlusive device of FIG. 4B, in accordance with embodiments of the present technology.

FIG. 4E is a top cross-sectional view of FIG. 4D.

FIG. 4F is a side cross-sectional view of the fully deployed occlusive device and coil of FIG. 4D, in accordance with embodiments of the present technology.

### DETAILED DESCRIPTION

The present technology relates to devices for treating vascular defects such as aneurysms, and associated systems and methods. In some embodiments, for example, an occlusive device for treating an aneurysm includes a first mesh (e.g., a first tubular braid) having a first petal-shaped region. The first mesh can be coupled to a second mesh (e.g., a second tubular braid) having a second petal-shaped region. When the occlusive device is deployed within the aneurysm, the first and second petal-shaped regions can extend at least partially over a neck of the aneurysm, thus providing mechanical support to retain an embolization coil within the aneurysm sac and prevent the coil from prolapsing into the parent vessel. Optionally, the petal-shaped regions can also engage a wall of the aneurysm to secure the occlusive device in place and prevent the device from being displaced by the pressure of the packed coils. Accordingly, the occlusive device can be advantageous for treating wide-necked aneurysms via coil embolization without requiring any implanted components in the parent vessel.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” and “lower” can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

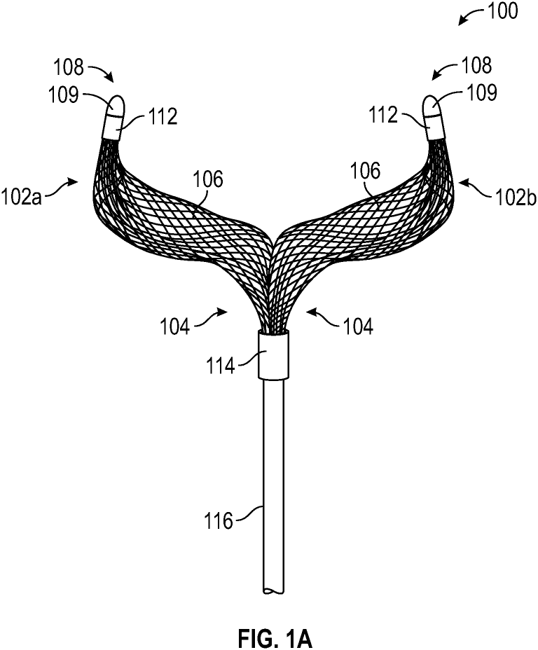

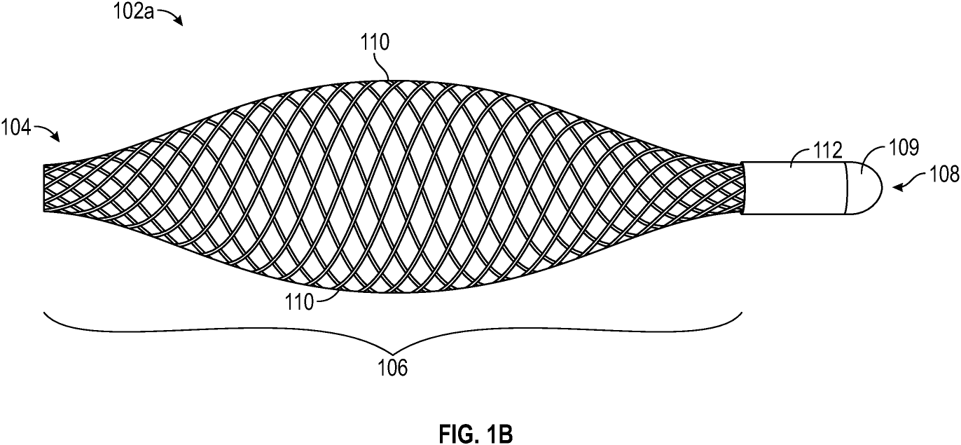

FIG. 1A is a perspective view of an occlusive device 100 (“device 100”) for treating an aneurysm configured in accordance with embodiments of the present technology. The device 100 includes a plurality of mesh structures configured to be deployed within an aneurysm sac. For example, as shown in FIG. 1A, the device 100 can include a first mesh structure 102a (“mesh 102a”) coupled to a second mesh structure 102b (“mesh 102b”). As described further below, the meshes 102a, 102b can be positioned over the neck of the aneurysm to prevent an embolization element (e.g., a coil) from protruding from the aneurysm sac into the parent vessel. The meshes 102a, 102b can also reduce or prevent blood flow from the parent vessel into the aneurysm sac, and/or provide a scaffold for endothelial cell attachment. The growth and development of an endothelial layer over the aneurysm neck can wall off the aneurysm from the parent vessel and allow flow dynamics to equilibrate at the defect. Upon endothelialization, the fluid pressure can be evenly distributed along the parent vessel in a manner that prevents recanalization at the defect post-treatment. Moreover, blood from within the parent vessel no longer has access to the walled-off defect once the endothelialization process is complete. Accordingly, the device 100 can facilitate healing of the defect and/or prevent recanalization.

Each mesh 102a, 102b is an elongated structure including a proximal end region 104, a petal-shaped region 106, and a distal end region 108. As shown in FIG. 1A, the meshes 102a, 102b are coupled to each other at the proximal end regions 104. The petal-shaped region 106 is a wider, flattened portion of each mesh 102a, 102b that extends distally from the proximal end region 104. As discussed further below, when the device 100 is deployed in the aneurysm, the petal-shaped regions 106 at least partially occlude the aneurysm neck. The petal-shaped regions 106 can also extend upward to engage the inner wall of the aneurysm sac to secure the device 100 in place. Each petal-shaped region 106 terminates in a respective distal end region 108. The distal end region 108 includes an atraumatic tip 109 having a rounded (e.g., hemispherical) shape.

In some embodiments, each of the meshes 102a, 102b is a tubular braid formed from a plurality of filaments, such as wires. At least some of the wires (e.g., at least 25%, 50%, 80%, 100% of the wires, etc.) can be made of one or more shape memory and/or superelastic materials (e.g., Nitinol). The braid can have, for example, from 44 to 144 wires, such as 64 or 72 wires. Some or all of the wires can have a diameter from 0.0010 inches to 0.0012 inches, such as a diameter of 0.0010 inches, 0.0011 inches, or 0.0012 inches (at least prior to etching). In some embodiments, some or all of the wires are drawn-filled tubes (“DFT”) having a radiopaque core (e.g., platinum) surrounded by a shape memory alloy and/or superelastic alloy (e.g., Nitinol, cobalt chromium, etc.). All or a portion of the length of some or all of the wires can have one or more coatings or surface treatments. For example, some or all of the wires can have a lubricious coating or treatment that reduces the delivery force of the meshes 102a, 102b as the device 100 is advanced through the delivery catheter. In some embodiments, the coating is relatively hydrophilic, such as a phosphorocholine compound. Additionally or alternatively, some or all of the wires can have a coating or treatment (the same as the lubricious coating, or a different coating) that enhances blood compatibility and reduces the thrombogenic surface activity of the braid. Optionally, at least a portion of the wires can be made of other suitable materials.

FIG. 1B is a top view of the first mesh 102a in a flattened state (the features of the second mesh 102b can be identical or generally similar to the features of the first mesh 102a, such that the following description of the first mesh 102a is also applicable to the second mesh 102b). As shown in FIG. 1B, in embodiments where the mesh 102a is a tubular braid, the petal-shaped region 106 can be a flattened section of the tubular braid, such that the petal-shaped region 106 includes two mesh layers that meet at folds at side edges 110 (only the top mesh layer is visible in FIG. 1B). The petal-shaped region 106 can have a generally oval shape, with the central portion of the petal-shaped region 106 being wider than the proximal and distal ends of the petal-shaped region 106. The proximal and/or distal end regions 104, 108 of the tubular braid can be closed (e.g., by welding, crimping, or other suitable techniques), or can remain open.

Optionally, the mesh 102a can include a collar 112 or similar structure (e.g., band, ring, wire) that is located at the distalmost portion of the petal-shaped region 106. In some embodiments, the tubular braid terminates within the collar 112, such that the distal end region 108 located distal to the collar 112 is made of a different material. For example, the tip 109 of the distal end region 108 can be made of a soft polymeric material (e.g., melted plastic, a plastic cap) that reduces the likelihood of inadvertently puncturing tissue. The collar 112 can be made of any suitable material, such as a metallic material. Optionally, the collar 112 can be made of a radiopaque material to facilitate visualization in vivo so the physician can confirm proper placement of the mesh 102a in the aneurysm. In embodiments where both of the meshes 102a, 102b include a respective collar 112, the collars 112 can be at different positions along the length of the meshes 102a, 102b from each other so that when the meshes 102a, 102b are loaded in a delivery catheter in a straightened, the collars 112 are offset from each other. This can be advantageous for reducing the size of the delivery catheter needed to accommodate the meshes 102a, 102b. In other embodiments, however, the collar 112 can be omitted from one or both of the meshes 102a, 102b.

Referring again to FIG. 1A, when the device 100 is in an expanded state, the meshes 102a, 102b can have a preset shape that is configured to conform to the inner contours of the aneurysm sac. For example, when the device 100 is fully expanded, the petal-shaped regions 106 can each have a curved (e.g., concave) surface having a radius of curvature similar to the radius of curvature of the corresponding portions of the aneurysm sac near the neck of the aneurysm. As shown in FIG. 1A, the curved surfaces of the petal-shaped regions 106 of the meshes 102a, 102b are both oriented to face generally upward. Accordingly, when the device 100 is deployed in the aneurysm, the curved surfaces of the petal-shaped regions 106 can face away from the aneurysm neck and toward the inner cavity of the aneurysm. The petal-shaped regions 106 can face the same direction (e.g., both face completely upward), or can face different directions. For example, as shown in FIG. 1A, the petal-shaped regions 106 are angled slightly toward each other such that the curved surfaces of the petal-shaped regions 106 slightly face each other. Additionally, the distal portions of the petal-shaped regions 106 can have an upwardly extending, curved shape that is biased slightly outward to engage with the aneurysm wall. For example, the distal portions of each petal-shaped region 106 can press against the aneurysm wall to exert a bracing force that prevents the device 100 from being displaced out of the aneurysm sac, as discussed further below. In other embodiments, however, the orientation and/or shape of the petal-shaped regions 106 can be varied as desired to conform to the contours of the aneurysm sac and/or support an embolization element within the aneurysm sac.

The meshes 102a, 102b are coupled to each other at the proximal end regions 104. For example, in the illustrated embodiment, the device 100 includes a band 114 (e.g., a ring, collar, wire, etc.) that physically attaches the proximal end regions 104 to each other. Alternatively or in combination, the proximal end regions 104 can be connected via adhesives, bonding, fasteners, and/or other attachment mechanisms. The band 114 can be made of any suitable material, such as a metallic material. Optionally, the band 114 can be made of a radiopaque material to facilitate visualization in vivo so the physician can confirm proper placement of the device 100 within the aneurysm.

The proximal end regions 104 of the meshes 102a, 102b can be coupled to a pusher member 116. The pusher member 116 can be an elongated rod, shaft, wire, etc., that is configured to push the device 100 through a distal end of a delivery catheter to deploy the device 100 within the aneurysm. Optionally, the pusher member 116 can also be used to pull the device 100 partially or fully back into the delivery catheter, e.g., for repositioning purposes. In some embodiments, the meshes 102a, 102b are detachably coupled to the pusher member 116 via a detachment element (not shown). The detachment element can utilize any suitable detachment technique known to those of skill in the art, such as electrolytic detachment, mechanical detachment, thermal detachment, electromagnetic detachment, or combinations thereof. An example of a detachment element for suitable use with the present technology is the Axium™ or Axium™ Prime Detachable Coil System (Medtronic).

The device 100 can be manufactured using various techniques. For example, in embodiments where the meshes 102a, 102b are formed from tubular braids, the petal-shaped regions 106 can be formed by wrapping a first portion of the tubular braid around a portion of a curved mold, then heat-setting the first portion to form the preset curved surface shown in FIG. 1A. In some embodiments, the tubular braid is wrapped less than 360° around a spherical mold having a radius of curvature equivalent to the radius of curvature of the resulting petal-shaped region 106. As the tubular braid is wrapped around the spherical mold, opposing portions of the tubular sidewall can be pressed toward one another along the length of the tubular braid, thereby flattening the tubular braid while conforming the braid to the curvature of the spherical mold. Depending on the geometry of the aneurysm to be treated, the petal-shaped region 106 can have other shapes or configurations and can be formed in a similar manner on molds having other shapes or sizes, such as non-spherical shapes, cylinders, hemispheres, polyhedrons (e.g., cuboids, tetrahedrons (e.g. pyramids), octahedrons, prisms, etc.), prolate spheroids, oblate spheroids, plates (e.g., discs, polygonal plates), bowls, non-spherical surfaces of revolution (e.g., toruses, cones, cylinders, or other shapes rotated about a center point or a coplanar axis), or combinations thereof.

Subsequently, the distal portions of the petal-shaped regions 106 can be cinched and/or enclosed in the collars 112. The tips 109 can then be attached to the petal-shaped regions 106 and/or collars 112, e.g., by bonding, melting, adhesives, interference fit, etc. The individual meshes 102a, 102b can then be coupled to each other to assemble the device 100.

The configuration of the device 100 shown in FIGS. 1A and 1B can be modified in many different ways. For example, although the meshes 102a, 102b are depicted as being generally similar to each other, in other embodiments, the meshes 102a, 102b can have different geometries, such as different lengths, widths, shapes, curvatures, etc. In some embodiments, the first mesh 102a has a larger petal-shaped region 106 than the second mesh 102b (e.g., with respect to surface area, length, width, etc.), or vice-versa. The shapes of the petal-shaped regions 106 can also be different in the first and second meshes 102a, 102b.

As another example, although the meshes 102a, 102b are illustrated as being separate structures, in other embodiments, the meshes 102a, 102b can be a single, unitary structure, such as a single tubular braid. In such embodiments, the tubular braid can include two flattened regions corresponding to the two petal-shaped regions 106. The tubular braid can include a folded region between the flattened regions that serves as the connection point for coupling to the band 114 and/or the pusher member 116.

In a further example, although the illustrated embodiment includes two meshes 102a, 102b, the device 100 can alternatively include a different number of meshes, such as three, four, five, or more meshes. Accordingly, the device 100 can include a total of three, four, five, or more petal-shaped regions 106. In such embodiments, the meshes can be circumferentially offset from each other in a spoke-like configuration, e.g., to provide more complete coverage of the aneurysm neck and/or distribute bracing forces more evenly around the circumference of the aneurysm. The meshes can all be identical or generally similar, or some or all of the meshes can have different geometries (e.g., lengths, widths, shapes, curvatures, etc.).

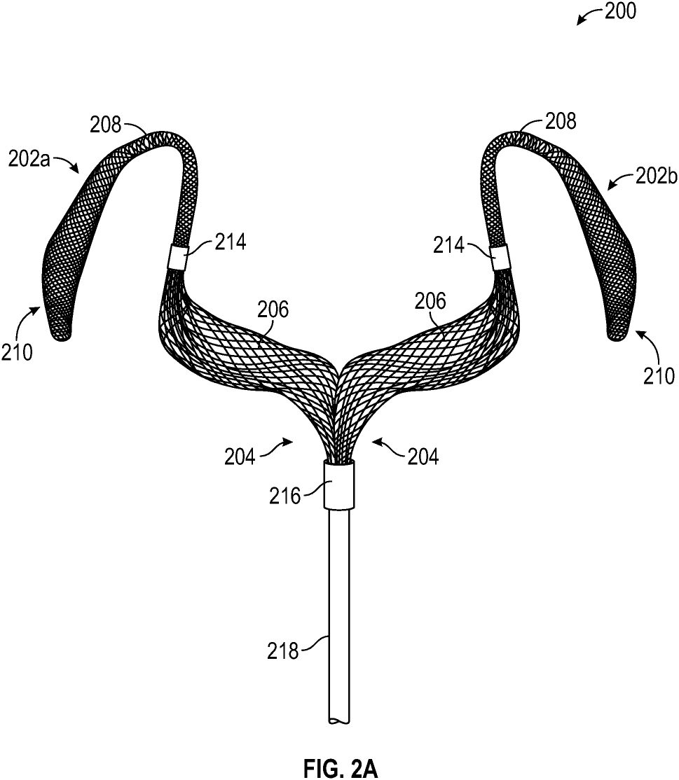

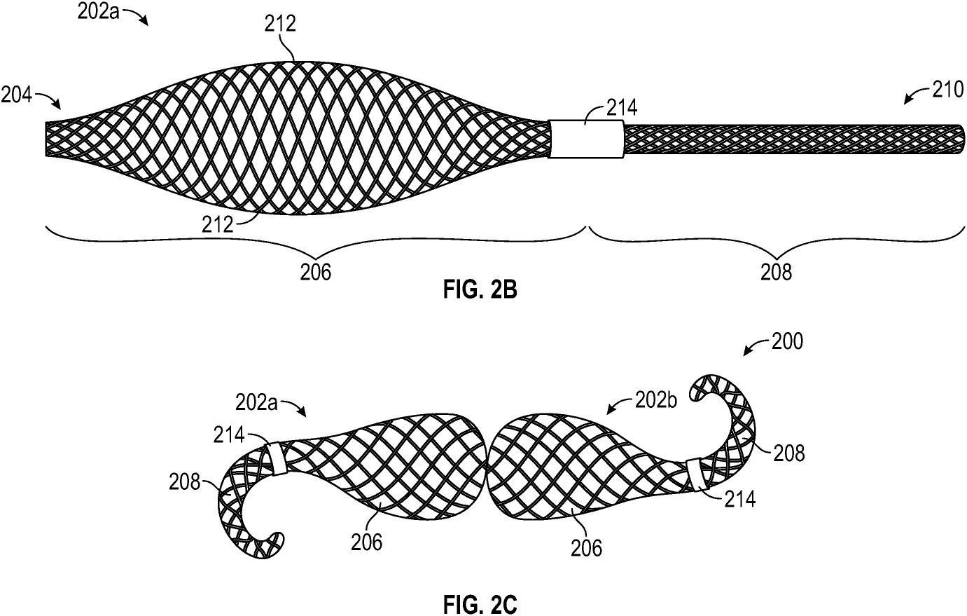

FIG. 2A is a perspective view of another occlusive device 200 (“device 200”) for treating an aneurysm configured in accordance with embodiments of the present technology. The device 200 includes a first mesh structure 202a (“mesh 202a”) and a second mesh structure 202b (“second mesh 202b”). Each mesh 202a, 202b is an elongated structure including a proximal end region 204, a petal-shaped region 206, an arm region 208, and a distal end region 210. The petal-shaped region 206 can be identical or generally similar to the petal-shaped region 106 of the device 100 of FIGS. 1A and 1B. For example, as shown in FIG. 2A, the petal-shaped region 206 is a wider, flattened portion of each mesh 202a, 202b that extends distally from the proximal end region 204 and is configured to at least partially occlude the aneurysm neck. The arm region 208 is a narrower, curved portion of the respective mesh 202a, 202b that extends distally from the petal-shaped region 206. The arm regions 208 are configured to engage the inner wall of the aneurysm sac to secure the device 200 in place. The arm region 208 terminates in the distal end region 108. Optionally, the arm regions 208 can also occupy some of the space within the aneurysm sac, similar to an embolization coil.

In the illustrated embodiment, each mesh 202a, 202b is a unitary structure such that the proximal end region 204, petal-shaped region 206, arm region 208, and distal end region 210 are continuous and integrally formed with each other. In other embodiments, one or both of the meshes 202a, 202b can formed from a plurality of discrete segments that are coupled to each other (e.g., by adhesives, bonding, fasteners). For example, each mesh 202a, 202b can include a first segment including the proximal end region 204 and the petal-shaped region 206, and the first segment can be coupled to a second segment including the arm region 208 and the distal end region 210. In some embodiments, each of the meshes 202a, 202b is a tubular braid, as discussed above.

FIG. 2B is a top view of the first mesh 202a in a flattened state (the features of the second mesh 202b can be identical or generally similar to the features of the first mesh 202a, such that the following description of the first mesh 202a is also applicable to the second mesh 202b). As shown in FIG. 2B, in embodiments where the mesh 202a is a tubular braid, the petal-shaped region 206 can be a flattened, radially expanded section of the tubular braid, such that the petal-shaped region 206 includes two mesh layers that meet at folds at side edges 212 (only the top mesh layer is visible in FIG. 2B). The petal-shaped region 206 can have a generally oval shape, with the central portion of the petal-shaped region 206 being wider than the proximal and distal ends of the petal-shaped region 206. The arm region 208 can be a narrower, radially compressed region of the tubular braid. The arm region 208 can have a tubular (e.g., cylindrical) shape, or can also be a flattened section with two folded mesh layers. The filaments of the petal-shaped region 206 can be spaced further apart from each other, while the filaments of the arm region 208 can remain more closely together. Accordingly, the petal-shaped region 206 can include larger interstices between filaments compared to the arm region 208. The proximal and/or distal end regions 204, 210 of the tubular braid can be closed (e.g., by welding, crimping, or other suitable techniques), or can remain open.

The dimensions of the petal-shaped region 206 and arm region 208 can be varied as desired. In some embodiments, for example, when the mesh 202a is in the flattened state, the petal-shaped region 206 constitutes at least 10%, 20%, 30%, 40%, 50%, 60%, 70%, 80%, or 90% of the total length of the mesh 202a, and/or the arm region 208 constitutes no more than 90%, 80%, 70%, 60%, 50%, 40%, 30%, 20%, or 10% of the total length of the mesh 202a. The ratio of the length of the petal-shaped region 206 to the length of the arm region 208 can be greater than or equal to 1:10, 1:5, 1:4, 1:3, 1:2, 1:1, 2:1, 3:1, 4:1, 5:1, or 10:1. In some embodiments, when the mesh 202a is in the flattened state, the width (e.g., maximum width) of the petal-shaped region 206 is at least 2 times, 3 times, 4 times, 5 times, 6 times, 7 times, 8 times, 9 times, or 10 times greater than the width (e.g., maximum width) of the arm region 208.

Optionally, the mesh 202a can include a collar 214 or similar structure (e.g., band, ring, wire) that is located between the petal-shaped region 206 and the arm region 208. The collar 214 can be identical or generally similar to the collar 112 of the device 100 of FIGS. 1A and 1B. In some embodiments, the collar 214 cinches the tubular braid to define the transition from the radially expanded petal-shaped region 206 to the radially compressed arm region 208. The collar 214 can also maintain the proximal end of the arm region 208 in a radially compressed state so that the filaments of the arm region 208 do not separate over time. In embodiments where both of the meshes 202a, 202b include a respective collar 214, the collars 214 can be at different positions along the length of the meshes 202a, 202b from each other so that when the meshes 202a, 202b are loaded in a delivery catheter in a straightened, the collars 214 are offset from each other. This can be advantageous for reducing the size of the delivery catheter needed to accommodate the meshes 202a, 202b. In other embodiments, however, the collar 214 can be omitted from one or both of the meshes 202a, 202b.

Referring again to FIG. 2A, when the device 200 is in an expanded state, the meshes 202a, 202b can have a preset shape that is configured to conform to the inner contours of the aneurysm sac. For example, when the device 200 is fully expanded, the petal-shaped regions 206 can each have a curved (e.g., concave) surface having a radius of curvature similar to the radius of curvature of the corresponding portions of the aneurysm sac near the neck of the aneurysm. The geometry of the petal-shaped regions 206 can be identical or generally similar to the geometry of the petal-shaped regions 106 of FIGS. 1A and 1B. When the device 200 is fully expanded, the arm regions 208 can each have a preset curved shape. In the illustrated embodiment, the arm regions 208 are curved downward, such that the distal end region 210 of each mesh 202a, 202b is located below the apex of the curve of the respective arm region 208. This curved shape can bias at least a portion of each arm region 208 outward and into engagement with the aneurysm wall. For example, the distal portions of each arm region 208 can press against the aneurysm wall to exert a bracing force that prevents the device 200 from being displaced out of the aneurysm sac, as discussed further below.

In some embodiments, the arm region 208 of each mesh 202a, 202b is curved in a different (e.g., opposite) direction than the petal-shaped region 206, such that each mesh 202a, 202b has a serpentine shape (e.g., an S shape). For example, the petal-shaped region 206 of the first mesh 202a can have a preset curve oriented in a first direction (e.g., generally concave up), and the arm region 208 of the first mesh 202a can have a preset curve oriented in a second, different direction (e.g., generally concave down). Similarly, the petal-shaped region 206 of the second mesh 202b can have a preset curve oriented in a third direction (e.g., generally concave up), and the arm region 208 of the second mesh 202b can have a preset curve oriented in a fourth direction (e.g., generally concave down). The collars 214 can be located at or near the inflection point of the serpentine shape.

Referring next to FIG. 2C, which illustrates a top view of the device 200 in the expanded state, the arm regions 208 can also be curved laterally, which can be advantageous for bracing the device 200 against the aneurysm wall. For example, when the device 200 is deployed in the aneurysm, the lateral curvature can allow the arm regions 208 to span at least 10%, 20%, 30%, 40%, 50%, 60%, 70%, 80%, or 90% of the circumference of the aneurysm. In the illustrated embodiments, for example, the arm region 208 of the first mesh 202a is curved in a first lateral direction, and the arm region 208 of the second mesh 202b is curved in a second lateral direction different from (e.g., opposite to) the first lateral direction. In other embodiments, however, the arm regions 208 of the meshes 202a, 202b can be curved in the same or similar lateral directions, or may not include any lateral curvature.

Referring again to FIG. 2A, the meshes 202a, 202b are coupled to each other at the proximal end regions 204. For example, in the illustrated embodiment, the device 200 includes a band 216 (e.g., a ring, collar, wire, etc.) that physically attaches the proximal end regions 204 to each other. The band 216 can be identical or generally similar to the band 114 of the device 100 of FIGS. 1A and 1B. Alternatively or in combination, the proximal end regions 204 can be connected via adhesives, bonding, fasteners, and/or other attachment mechanisms.

The proximal end regions 204 of the meshes 202a, 202b can be coupled to a pusher member 218, which can be identical or generally similar to the pusher member 116 of the device 100 of FIGS. 1A and 1B. In some embodiments, the meshes 202a, 202b are detachably coupled to the pusher member 218 via a detachment element (not shown). The detachment element can utilize any suitable detachment technique known to those of skill in the art, such as electrolytic detachment, mechanical detachment, thermal detachment, electromagnetic detachment, or combinations thereof, as discussed above.

The device 200 can be manufactured using various techniques. For example, in embodiments where the meshes 202a, 202b are formed from tubular braids, the petal-shaped regions 206 can be formed by wrapping a first portion of the tubular braid around a portion of a curved mold, then heat-setting the first portion to form the preset curved surface, as described above with respect to the petal-shaped regions 106 of FIGS. 1A and 1B. The arm regions 208 can be formed by pinching, flattening, or otherwise compacting a second portion of the tubular braid to form the radially compressed sections. The radially compressed sections can then be heat set into the desired curved shape using a suitably shaped mold and/or other techniques known to those of skill in the art. The arm region 208 can be formed before, after, or concurrently with the petal-shaped region 206. The individual meshes 202a, 202b can then be coupled to each other to assemble the device 200.

The configuration of the device 100 shown in FIGS. 2A-2C can be modified in many different ways. For example, although the meshes 202a, 202b are depicted as being generally similar to each other, in other embodiments, the meshes 202a, 202b can have different geometries, such as different lengths, widths, shapes, curvatures, etc. In some embodiments, the first mesh 202a has a larger petal-shaped region 206 than the second mesh 202b (e.g., with respect to surface area, length, width, etc.), or vice-versa. Similarly, the first mesh 202a can have a longer or shorter arm region 208 than the second mesh 202b. The shapes of the petal-shaped regions 206 and/or arm regions 208 can also be different in the first and second meshes 202a, 202b.

As another example, although the meshes 202a, 202b are illustrated as being separate structures, in other embodiments, the meshes 202a, 202b can be a single, unitary structure, such as a single tubular braid. In such embodiments, the tubular braid can include two flattened regions corresponding to the two petal-shaped regions 206, and two radially compressed regions corresponding to the two arm regions 208. The tubular braid can include a folded region between the flattened regions that serves as the connection point for coupling to the band 216 and/or the pusher member 218.

In a further example, although the illustrated embodiment includes two meshes 202a, 202b, the device 200 can alternatively include a different number of meshes, such as three, four, five, or more meshes. Accordingly, the device 200 can include a total of three, four, five, or more petal-shaped regions 206, and a total of three, four, five, or more arm regions 208. In such embodiments, the meshes can be circumferentially offset from each other in a spoke-like configuration, e.g., to provide more complete coverage of the aneurysm neck and/or distribute bracing forces more evenly around the circumference of the aneurysm. The meshes can all be identical or generally similar, or some or all of the meshes can have different geometries (e.g., lengths, widths, shapes, curvatures, etc.).

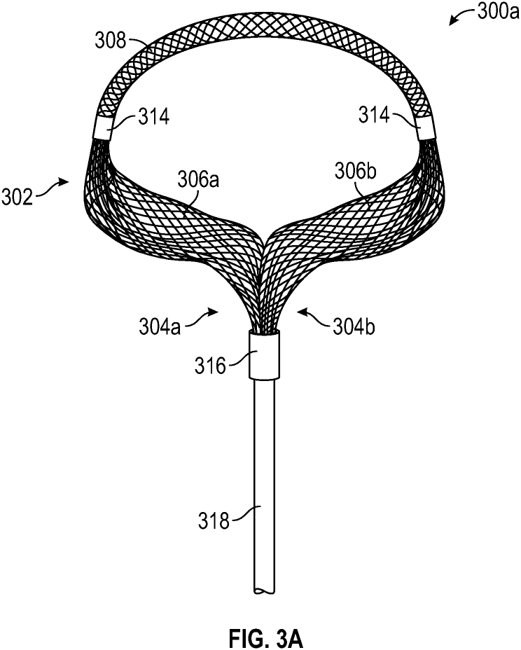

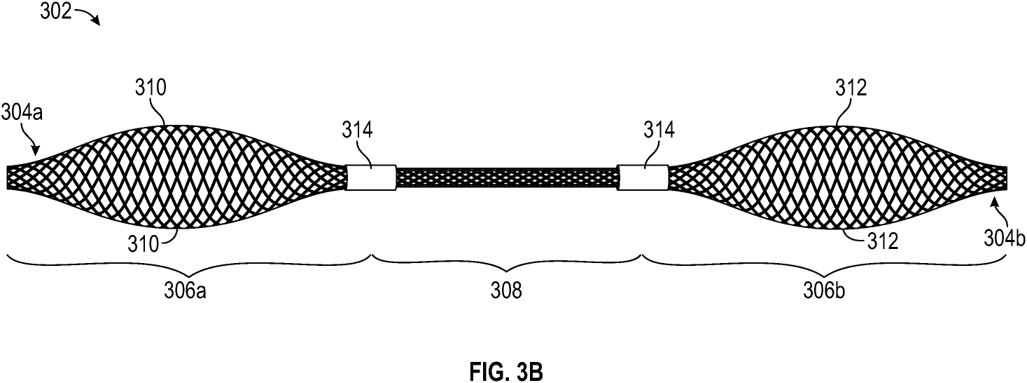

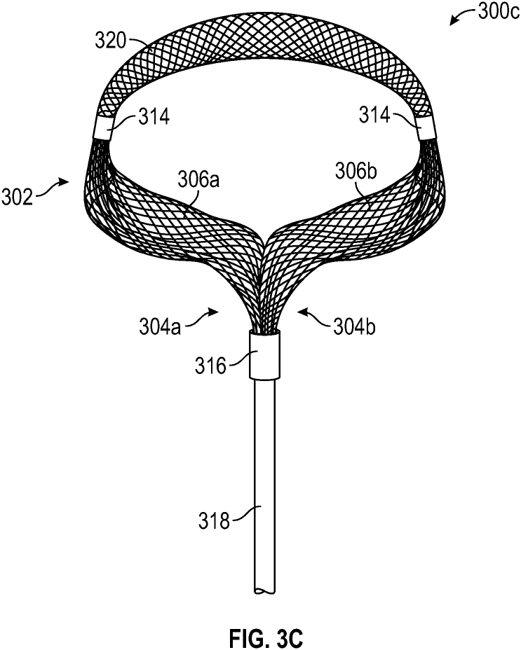

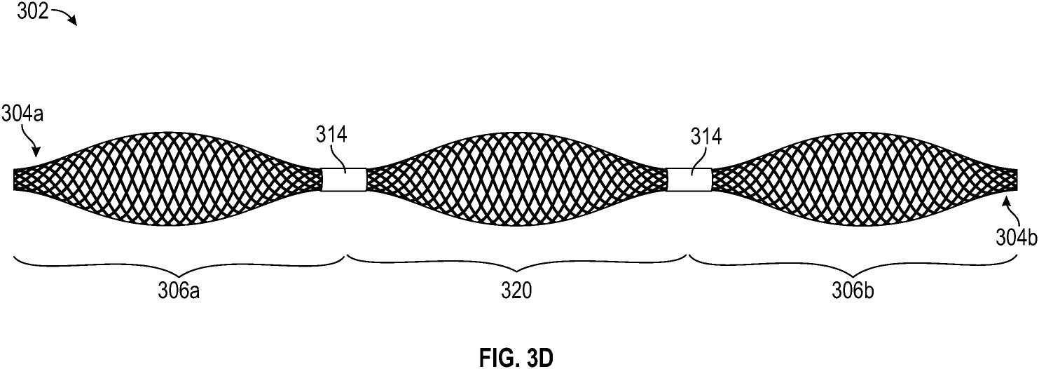

FIG. 3A is a perspective view of another occlusive device 300a (“device 300a”) for treating an aneurysm configured in accordance with embodiments of the present technology. The device 300a includes a single mesh structure 302 (“mesh 302”). The mesh 302 is an elongated structure extending from a first end region 304a to a second end region 304b. As shown in FIG. 3A, the mesh 302 includes a first petal-shaped region 306a, a second petal-shaped region 306b, and a bridge region 308. The first end region 304a is connected to the first petal-shaped region 306a, which connects to the bridge region 308, which connects to the second petal-shaped region 306b, which terminates in the second end region 304b. The petal-shaped regions 306a, 306b can be identical or generally similar to the petal-shaped regions previously described herein with respect to FIGS. 1A-2C. For example, as shown in FIG. 2A, the petal-shaped regions 306a, 306b are wider, flattened portions of the mesh 302 that are configured to at least partially occlude the aneurysm neck. The bridge region 308 is a narrower, curved portion of the mesh 302 that connects the petal-shaped regions 306a, 306b. The bridge region 308 is configured to engage the inner wall of the aneurysm sac to secure the device 300a in place. In some embodiments, the bridge region 308 braces against the dome of the aneurysm to stabilize the device 300a within the aneurysm sac. The mesh 302 can be a unitary structure such that the end regions 304a, 304b, petal-shaped regions 306a, 306b, and bridge region 308 are continuous and integrally formed with each other. In other embodiments, the mesh 302 can be formed from a plurality of discrete segments that are coupled to each other (e.g., by adhesives, bonding, fasteners). In some embodiments, the mesh 302 is a tubular braid, as discussed above.

FIG. 3B is a top view of the mesh 302 in a flattened state. As shown in FIG. 3A, in embodiments where the mesh 302 is a tubular braid, each petal-shaped region 306a, 306b is a flattened, radially expanded section of the tubular braid, such that each petal-shaped region 306a, 306b includes two mesh layers that meet at folds at side edges 310, 312, respectively. Each petal-shaped region 306a, 306b can have a generally oval shape, with the central portion being wider than the ends. The bridge region 308 can be a narrower, radially compressed region of the tubular braid. The bridge region 308 can have a tubular (e.g., cylindrical) shape, or can also be a flattened section with two folded mesh layers. The filaments of the petal-shaped region 306a, 306b can be spaced further apart from each other compared to the filaments of the bridge region 308. The first and second end regions 304a, 304b of the tubular braid can be closed (e.g., by welding, crimping, or other suitable techniques), or can remain open.

The dimensions of the petal-shaped regions 306a, 306b and the bridge region 308 can be varied as desired. For example, in some embodiments, the first petal-shaped region 306a has the same geometry (e.g., length, width, shape) as the second petal-shaped region 306b. Alternatively, the first petal-shaped region 306a can be longer or shorter than the second petal-shaped region 306b, can be wider or narrower than the second petal-shaped region 306b, and/or can have a different shape than the second petal-shaped region 306b. When the mesh 302 is in the flattened state, the petal-shaped regions 306a, 306b can collectively constitute at least 10%, 20%, 30%, 40%, 50%, 60%, 70%, 80%, or 90% of the total length of the mesh 302, and/or the bridge region 308 can constitute no more than 90%, 80%, 70%, 60%, 50%, 40%, 30%, 20%, or 10% of the total length of the mesh 302. The ratio of the combined length of the petal-shaped regions 306a, 306b to the length of the bridge region 308 can be greater than or equal to 1:10, 1:5, 1:4, 1:3, 1:2, 1:1, 2:1, 3:1, 4:1, 5:1, or 10:1. In some embodiments, when the mesh 302 is in the flattened state, the width (e.g., maximum width) of each petal-shaped region 306a, 306b is at least 2 times, 3 times, 4 times, 5 times, 6 times, 7 times, 8 times, 9 times, or 10 times greater than the width (e.g., maximum width) of the bridge region 308.

Optionally, the mesh 302 can include at least one collar 314 (e.g., band, ring, wire). For example, the mesh 302 can include a first collar 314 located between the first petal-shaped region 306a and the bridge region 308, and a second collar 314 located between the second petal-shaped region 306b and the bridge region 308. The collars 314 can be identical or generally similar to the collar 112 of the device 100 of FIGS. 1A and 1B. In some embodiments, the collars 314 cinch the tubular braid to define the transition from the radially expanded petal-shaped regions 306a, 306b to the radially compressed bridge region 308. The collars 314 can also maintain the ends of the bridge region 308 in a radially compressed state so that the filaments of the bridge region 308 do not separate over time.

Referring again to FIG. 3A, the end regions 304a, 304b of the mesh 302 are coupled to each other by a band 316 (e.g., a ring, collar, wire, etc.), which can be identical or generally similar to the band 114 of the device 100 of FIGS. 1A and 1B. Alternatively or in combination, the end regions 304a, 304b can be connected via adhesives, bonding, fasteners, and/or other attachment mechanisms. The pend regions 304a, 304b can be coupled to a pusher member 318, which can be identical or generally similar to the pusher member 116 of the device 100 of FIGS. 1A and 1B. In some embodiments, the meshes 202a, 202b are detachably coupled to the pusher member 218 via a detachment element (not shown). The detachment element can utilize any suitable detachment technique known to those of skill in the art, such as electrolytic detachment, mechanical detachment, thermal detachment, electromagnetic detachment, or combinations thereof, as discussed above.

The device 300a can be manufactured using various techniques. For example, in embodiments where the mesh 302 is formed from tubular braids, the petal-shaped regions 306a, 306b can be formed by wrapping respective portions of the tubular braid around a curved mold, then heat-setting the respective portion to form the preset curved surface, as previously described. The bridge region 308 can be formed by pinching, flattening, or otherwise compacting another portion of the tubular braid to form the radially compressed sections. The radially compressed sections can then be heat set into the desired curved shape using a suitably shaped mold and/or other techniques known to those of skill in the art. The bridge region 308 can be formed before, after, or concurrently with the petal-shaped regions 306a, 306b. The end regions 304a, 304b can then be coupled to each other to form the device 300a.

FIG. 3C is a perspective view of another occlusive device 300c (“device 300c”) for treating an aneurysm configured in accordance with embodiments of the present technology. The device 300c can be generally similar to the device 300a of FIGS. 3A and 3B, except that the device 300c includes a wider, expanded bridge region 320. Accordingly, the discussion of the device 300c will be limited to those features that differ from the device 300a of FIGS. 3A and 3B.

The bridge region 320 can be similar to the petal-shaped regions 306a, 306b in that it is a wider, flattened portion of the mesh 302. For example, as best seen in FIG. 3D, which illustrates a top view of the mesh 302 in a flattened state, in embodiments where the mesh 302 is a tubular braid, the bridge region 308 can be a flattened, radially expanded section of the tubular braid with two mesh layers connected at side edges. This configuration of the bridge region 320 can increase the surface area of the mesh 302 that contacts and braces against the dome of the aneurysm, thus further stabilizing the device 300c within the aneurysm sac.

The geometry (e.g., shape, dimensions) of the bridge region 320 can be varied as desired. For example, when fully expanded, the bridge region 320 can have a hemispherical shape, a half-hemispherical shape, or any other shape suitable for conforming to the inner contours of the aneurysm sac. Although FIG. 3D illustrates the bridge region 320 as having the same geometry (e.g., length, width, shape) as the first and second petal-shaped regions 306a, 306b, in other embodiments, the bridge region 320 can have a different geometry than the first and/or second petal-shaped regions 306a, 306b, e.g., with respect to length, width, shape, etc. The bridge region 320 can be formed using the same or similar techniques used to form the petal-shaped regions 306a, 306b, as described elsewhere herein.

The configuration of the devices 300a, 300c shown in FIGS. 3A-3D can be modified in many different ways. For example, although the illustrated embodiments each depict a single mesh 302, any of the devices 300a, 300c can alternatively include a different number of meshes, such as two, three, four, five, or more meshes. Accordingly, the devices 300a, 300c can include a total of four, six, eight, ten, or more petal-shaped regions, and a total of two, three, four, five, or more bridge regions. In such embodiments, the meshes can be circumferentially offset from each other in a spoke-like configuration, e.g., to provide more complete coverage of the aneurysm neck and/or distribute bracing forces more evenly around the circumference of the aneurysm. The meshes can all be identical or generally similar, or some or all of the meshes can have different geometries (e.g., lengths, widths, shapes, curvatures, etc.).

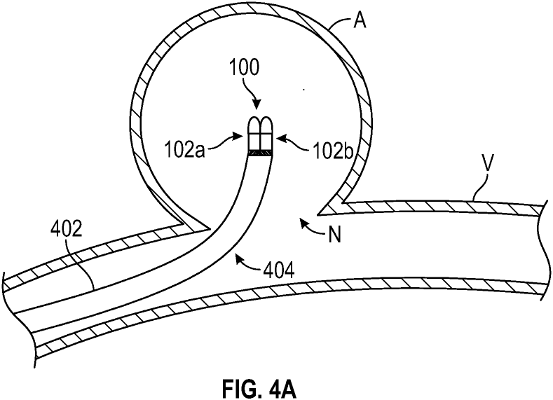

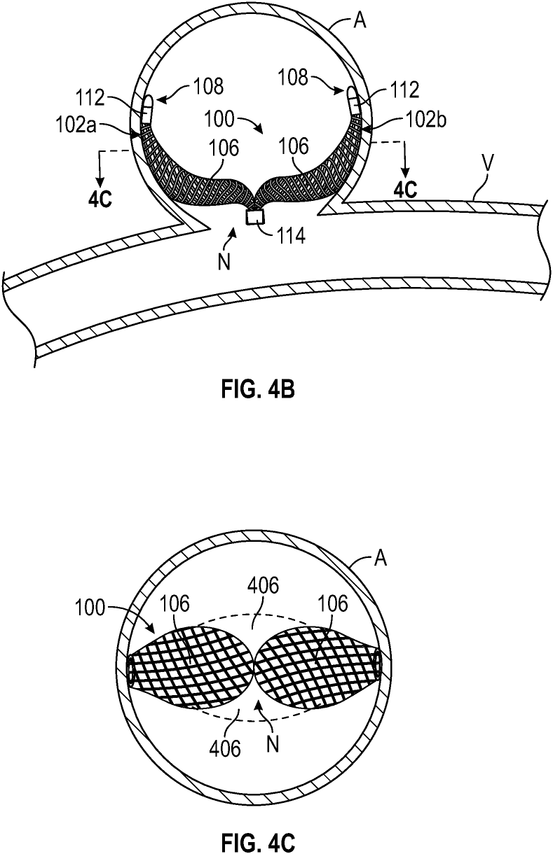

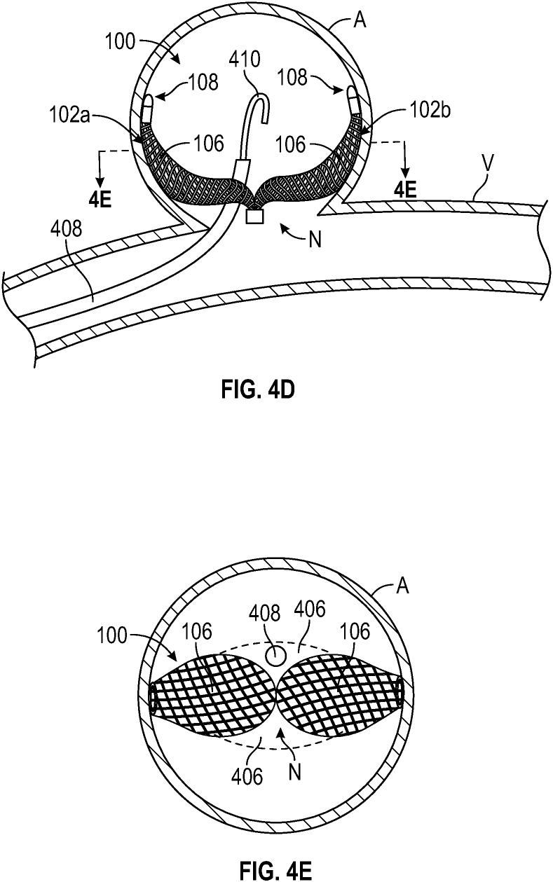

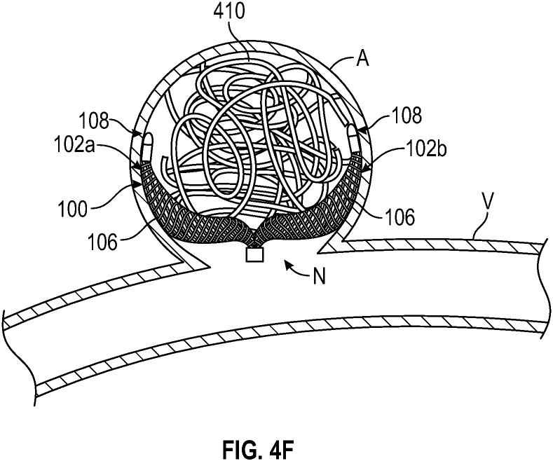

FIGS. 4A-4F illustrate a method of deploying an occlusive device, in accordance with embodiments of the present technology. Although the illustrated embodiment is shown and described in terms of the device 100 of FIGS. 1A and 1B, the method can be applied to any embodiment of the occlusive devices described herein. Referring first to FIG. 4A, the device 100 can be loaded within a first elongated member 402 (e.g., a delivery catheter such as a microcatheter) in a low-profile state (not visible in FIG. 2A). When in the low-profile state, the meshes 102a, 102b of the device 100 can be compressed, flattened, or otherwise compacted in a generally linear configuration to conform to the interior lumen of the first elongated member 402. The device 100 can then be intravascularly delivered to a location within a blood vessel V adjacent a target aneurysm A via the first elongated member 402. As shown in FIG. 4A, a distal tip portion 404 of the first elongated member 402 can be advanced through the neck N of the aneurysm A and into an interior cavity of the aneurysm A. The device 100 can then be deployed by pushing the device 100 (e.g., using the pusher member 116 of FIG. 1A) distally through the distal opening of the first elongated member 402 and into the aneurysm cavity. As shown in FIG. 4A, as the meshes 102a, 102b of the device 100 exit the first elongated member 402, they can self-expand into their preset curved shapes.

Referring next to FIG. 4B, once the device 100 is fully deployed and has assumed its expanded state, the device 100 can be detached (e.g., using the detachment techniques described above), and the first elongated member 402 can be withdrawn from the aneurysm A. When the device 100 is fully deployed, the petal-shaped regions 106 of the device 100 can be positioned adjacent to and over the aneurysm neck N. As best seen in FIG. 4C (showing a cross-sectional view of FIG. 4B), the petal-shaped regions 106 can only partially occlude the aneurysm neck N, thus leaving one or more gaps 306 providing a passageway from the vessel V into the aneurysm A. The area of the gaps 306 can collectively be no more than 75%, 50%, 40%, 30%, 20%, or 10% of the total area of the neck N. Although FIG. 4C illustrates two gaps 306, one at each side of the device 100, in other embodiments, the device 100 can be configured to leave only a single gap 306 at one side of the device 100.

Referring again to FIG. 4A, the distal portions of the petal-shaped regions 106 can extend away from the aneurysm neck N and into the aneurysm cavity. As previously discussed, the curvature of the petal-shaped regions 106 can bias these components into engagement with the interior wall, thus generating a bracing force against the aneurysm wall that prevents the device 100 from being displaced out of the aneurysm A and into the vessel V. In embodiments where the device includes arm regions distal to the petal-shaped regions (e.g., the device 200 of FIGS. 2A-2C), the arm regions can brace against the aneurysm wall, alternatively or in addition to the petal-shaped regions. In some embodiments, when the device 100 is fully deployed, the device 100 is located entirely within the aneurysm sac and does not include any components that protrude into the vessel V. Alternatively, the marker band 114 may protrude partially into the vessel V, but the meshes 102a, 102b can be located entirely within the aneurysm sac. This approach can be advantageous for reducing disruptions to blood flow in the vessel V, which may lead to thrombus formation.

Referring next to FIG. 4D, a second elongated member 408 (e.g., a second delivery catheter, such as a microcatheter) can subsequently be used to introduce an embolization coil 410 or other embolization element into the aneurysm A. As best seen in FIG. 4E (showing a cross-sectional view of FIG. 4D), the second elongated member 408 can be advanced through one of the gaps 406a between the petal-shaped regions 106 and the aneurysm neck N, and into the interior of the aneurysm A. The coil 410 can then be deployed out of the distal opening of the second elongated member 408 and into the aneurysm A. In other embodiments, however, the first elongated member 402 can be used to introduce both the device 100 and the coil 410 into the aneurysm.

Referring next to FIG. 4F, once fully deployed, the coil 410 can fill most or substantially all of the space within the aneurysm A. The coil 410 can be formed of one or more wires wound in a helical fashion about an axis to form an elongated tubular member. The wire(s) forming the coil 410 can be circular, square, or rectangular in cross-section, and can have a cross-sectional dimension (e.g., width, radius) from 0.001 inches to 0.003 inches, or from 0.0015 inches to 0.0025 inches. In some embodiments, the wire(s) forming the coil 410 have a cross-sectional dimension no greater than 0.003 inches, 0.0025 inches, or 0.002 inches. The coil 410 can be circular, square, or rectangular in cross-section, and can have a cross-sectional dimension (e.g., width, radius) from 0.01 inches to 0.02 inches, from 0.012 inches to 0.018 inches, or from 0.014 inches to 0.016 inches. In some embodiments, the coil 410 has a cross-sectional dimension that is no greater than 0.0145 inches or 0.0140 inches.

The coil 410 can have a length from 2 cm to 30 cm, from 3 cm to 25 cm, or from 4 cm to 20 cm. In some embodiments, the length of the coil 410 depends on the size of the aneurysm being treated. For example: for an aneurysm 4 mm in diameter or less, the coil 410 can have a length of about 6 cm; for an aneurysm 5 mm in diameter or less, the coil 410 can have a length of about 8 cm; for an aneurysm 6 mm in diameter or less, the coil 410 can have a length of about 15 cm; for an aneurysm 7 mm in diameter or less, the coil 410 can have a length of about 15 cm; for an aneurysm 8 mm in diameter or less, the coil 410 can have a length of about 20 cm; and, for an aneurysm 9 mm in diameter or less, the coil 410 can have a length of about 20 cm.

The coil 410 can be made from metals, alloys, polymers, shape memory materials (e.g., Nitinol), platinum, rhodium, palladium, tungsten, gold, silver, cobalt-chromium, platinum tungsten, and/or various alloys of these materials. In some embodiments, the coil 410 is heat set to form a tertiary structure (e.g., a pre-determined three-dimensional structure) when in a deployed state. For example, the coil 410 can have a preset tertiary structure that biases the coil into a bundled or more globular state that facilitates positioning of the coil 410 between the deployed device 100 and the aneurysm wall. In other embodiments, however, the coil 410 may not have a tertiary structure.

As previously mentioned, embolic coils such as the coil 410 can be very effective at filling space within the aneurysm cavity. However, there is a risk that the coil 410 may prolapse through the neck N of the aneurysm A into the vessel V, particularly if the aneurysm A is a wide-necked aneurysm (e.g., having a neck diameter greater than 4 mm and/or a dome-to-neck ratio less than 2). The device 100 can address this challenge via the petal-shaped regions 106 that are positioned over the aneurysm neck N to support the coil 410 and prevent the coil 410 from protruding into the neck, while the arm regions 118 brace against the aneurysm wall to resist the outward pressure toward the vessel V exerted by the packed coil 410 so the device 100 does not bulge into the vessel V.

The methods of the present technology can be performed under fluoroscopy such that the radiopaque portions of the device 100 (e.g., the collars 112 and/or band 114) can be visualized by the physician to ensure proper neck coverage. If the device 100 is not positioned properly, the physician can withdraw the device 100 into the first elongated member 402, reposition, and deploy again. Additionally, in embodiments where the coil 410 is radiopaque, the physician can use fluoroscopy to confirm that the coil 410 does not protrude from the neck N of the aneurysm A after deployment.

### Conclusion

Although many of the embodiments are described above with respect to systems, devices, and methods for treating a cerebral aneurysm, the technology is applicable to other applications and/or other approaches. For example, the occlusive devices, systems, and methods of the present technology can be used to treat any vascular defect and/or fill or partially fill any body cavity or lumen or walls thereof, such as to treat parent vessel occlusion, endovascular aneurysms outside of the brain, arterial-venous malformations, embolism, atrial and ventricular septal defects, patent ductus arteriosus, and patent foramen ovale. Additionally, several other embodiments of the technology can have different states, components, or procedures than those described herein. It will be appreciated that specific elements, substructures, advantages, uses, and/or other features of the embodiments described can be suitably interchanged, substituted or otherwise configured with one another in accordance with additional embodiments of the present technology. A person of ordinary skill in the art, therefore, will accordingly understand that the technology can have other embodiments with additional elements, or the technology can have other embodiments without several of the features shown and described above with reference to FIGS. 1A-4F.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1-69. (canceled)

70. An occlusive device for treating an aneurysm, the occlusive device comprising:
a first mesh formed from a first tubular braid, the first mesh including a first petal-shaped region formed from a first flattened section of the first tubular braid; and
a second mesh coupled to the first mesh, the second mesh formed from a second tubular braid, the second mesh including a second petal-shaped region formed from a second flattened section of the second tubular braid,
wherein, when the occlusive device is deployed within the aneurysm, the first and second petal-shaped regions are configured to extend at least partially over a neck of the aneurysm.

71. The occlusive device of claim 70, wherein the first mesh comprises a first tip connected to the first petal-shaped region, and the second mesh comprises a second tip connected to the second petal-shaped region.

72. The occlusive device of claim 71, wherein the first and second tips are rounded.

73. The occlusive device of claim 70, wherein the first mesh comprises a first arm region connected to the first petal-shaped region, and the second mesh comprises a second arm region connected to the second petal-shaped region.

74. The occlusive device of claim 70, wherein the first and second tubular braids are the same tubular braid.

75. The occlusive device of claim 74, further comprising a bridge region connecting the first petal-shaped region and the second petal-shaped region.

76. The occlusive device of claim 70, wherein:
the first petal-shaped region includes a proximal portion configured to cover the neck of the aneurysm, and a distal portion configured to engage a wall of the aneurysm, and
the second petal-shaped region includes a proximal portion configured to cover the neck of the aneurysm, and a distal portion configured to engage the wall of the aneurysm.

77. The occlusive device of claim 70, wherein the first mesh includes a first proximal end region connected to the first petal-shaped region, the second mesh includes a second proximal end region connected to the second petal-shaped region, and the first and second meshes are coupled to each other at the first and second proximal end regions.

78. The occlusive device of claim 70, wherein the first petal-shaped region has a preset curve oriented in a first direction, and the second petal-shaped region has a preset curve oriented in a second direction.

79. The occlusive device of claim 78, wherein the preset curves of the first and second petal-shaped regions are configured to bias at least a portion of the first and second petal-shaped regions against a wall of the aneurysm.

80. The occlusive device of claim 70, further comprising a detachment element configured to releasably couple the first and second meshes to a pusher member.

81. The occlusive device of claim 70, further comprising at least one third mesh coupled to the first and second meshes, the at least one third mesh formed from at least one third tubular braid, the at least one third mesh including a third petal-shaped region formed from a third flattened section of the at least one third tubular braid.

82. The occlusive device of claim 70, wherein the first and second meshes each have a low-profile state for intravascular delivery to the aneurysm, and an expanded state for deployment within the aneurysm.

83. An occlusive device for treating an aneurysm, the occlusive device comprising:
a first mesh including a first petal-shaped region and a first arm region, the first arm region having a curved shape that extends distally from the first petal-shaped region; and
a second mesh coupled to the first mesh, the second mesh including a second petal-shaped region and a second arm region, the second arm region having a curved shape that extends distally from the second petal-shaped region,
wherein, when the occlusive device is deployed within the aneurysm, the first and second petal-shaped regions are configured to extend at least partially over a neck of the aneurysm, and the first and second arm regions are configured to engage a wall of the aneurysm.

84. The occlusive device of claim 83, wherein:
the first petal-shaped region has a preset curve oriented in a first direction, and the first arm region has a preset curve oriented in a second direction different from the first direction, and
the second petal-shaped region has a preset curve oriented in a third direction, and the second arm region has a preset curve oriented in a fourth direction different from the third direction.

85. The occlusive device of claim 83, wherein, when the occlusive device is deployed within the aneurysm, the first and second petal-shaped regions each define a concave surface facing away from the neck of the aneurysm.

86. The occlusive device of claim 83, wherein, when the occlusive device is deployed within the aneurysm, the curved shape of the first arm region biases a distal portion of the first arm region against the wall of the aneurysm, and the curved shape of the second arm region biases a distal portion of the second arm region against the wall of the aneurysm.

87. An occlusive device for treating an aneurysm, the device comprising:
a first tubular braid including a first flattened region connected to a first radially compressed region, the first radially compressed region having a preset curve; and
a second tubular braid coupled to the first tubular braid, the second tubular braid including a second flattened region connected to a second radially compressed region, the second radially compressed region having a preset curve,
wherein, when the occlusive device is deployed within the aneurysm, the first and second flattened regions are configured to at least partially cover a neck of the aneurysm, and the first and second radially compressed regions are biased into engagement with the wall of aneurysm by their respective preset curves.

88. The occlusive device of claim 87, wherein, when the occlusive device is deployed within the aneurysm, the first and second flattened regions each have a concave surface facing away from the neck of the aneurysm.

89. The occlusive device of claim 87, wherein the first flattened region has a preset curve oriented in a different direction than the preset curve of the first radially compressed region, and the second flattened region has a preset curve oriented in a different direction than the preset curve of the second radially compressed region.
